(19) United States
(12) Patent Application Publication
(10) Pub. No.: US 2016/0166489 A1
(43) Pub. Date: Jun. 16, 2016

(54) GEL-BASED SUGAR SCRUB

(71) Applicant: Mary Kay Inc., Addison, TX (US)

(72) Inventor: Mauricio CASTRO, Addison, TX (US)

(21) Appl. No.: 14/959,310

(22) Filed: Dec. 4, 2015

Related U.S. Application Data
(60) Provisional application No. 62/090,630, filed on Dec. 11, 2014.

(51) Int. Cl. A61K 8/60; A61Q 19/00; A61Q 19/10

(52) U.S. Cl. CPC A61K 8/60; A61Q 19/10; A61Q 19/007; A61K 2800/40

(57) ABSTRACT

Disclosed is a stable gel comprising a gel matrix comprising polyethylene glycol and surface treated silica, wherein the gel matrix is formed with the polyethylene glycol and surface treated silica, and particulate sugar dispersed throughout the gel matrix, wherein the gel has less than 5 wt. % oil.

### CROSS REFERENCE TO RELATED APPLICATIONS

This application claims benefit to U.S. Provisional Patent Application No. 62/090,630, titled “GEL-BASED SUGAR SCRUB”, filed Dec. 11, 2014. The entire contents of the referenced application is incorporated herein by reference without disclaimer.

### BACKGROUND OF THE INVENTION

A. Field of the Invention

The present invention relates generally to oil-free sugar scrubs that can be used to exfoliate, cleanse, or moisturize skin. The sugar scrubs are structured as gels that have granulated or particulate sugar evenly dispersed throughout a gel matrix.

B. Description of Related Art

Several skin moisturizing and/or exfoliating compositions containing sugar or other exfoliating agents are currently available. These compositions have various drawbacks ranging from unpleasant tactile properties (e.g., heavy, greasy, or sticky feel), instability issues, skin-irritation issues, or insufficient moisturizing capabilities.

The stability issue results from the sugar coalescing together and presenting as streaks or “caking” while being stored or during application to skin. U.S. Patent Application No. 2012/0225105 describes a stable anhydrous dispersion that includes a sugar phase that includes a continuous oil phase and a combination of granulated sugar and powdered sugar. While this composition has improved stability, the stability is derived in part from the use of a high amount of oil. In particular, the oil-phase is the continuous phase of the composition, which results in a “heavier” or “oily” feel when being topically applied to skin.

### SUMMARY OF THE INVENTION

The present invention overcomes deficiencies in the art by providing a gel-based sugar scrub having a gel matrix and granulated or particulate sugar dispersed throughout the gel matrix. The gel matrix is structured such that the gel is easily spreadable on skin while containing high amounts of particulate sugar. The sugar remains evenly dispersed throughout the gel matrix (i.e., no caking or coalescence of the sugar phase is observed under standard storage conditions such as 15 to 30° C.). The gel matrix is created by the interaction of surface treated silica particles (e.g., silica silylate) with polyethylene glycol molecules. Without wishing to be bound by theory, it is believed that this interaction results in a gel-matrix that allows for increased amounts of sugar to remain suspended or dispersed throughout the gel matrix. This is achieved without the use of oils (e.g., vegetable oils, mineral oils, petrolatum-based oils), thereby imparting a lighter or less oily feel when compared to currently available sugar scrubs. Further, the “lighter” feel is obtained without using silicone-based oils (e.g., cyclomethicone, dimethicone, etc.).

In one aspect, a gel that includes a gel matrix that can include polyethylene glycol and surface treated silica and particulate sugar dispersed throughout the gel matrix is disclosed. The gel matrix is formed from the polyethylene glycol and surface treated silica. The gel can include less than 5 wt. % of oil and less than 5 wt. % of water. In a particular aspect, the gel includes 15 wt. % to 40 wt. % of polyethylene glycol and 1 wt. % to 5 wt. % of surface treated silica. Still further, the gel can include 30 wt. % to 70 wt. % of particulate sugar. In another aspect, the gel can include 20 wt. % to 30 wt. % of polyethylene glycol, 1 wt. % to 3 wt. % of surface treated silica, and 35 wt. % to 50 wt. % of particulate sugar. The gel can further including a rheological modifier to modify the viscosity of the gel. In one instance, the rheological modifier can be a sugar alcohol (e.g., glycerol) and can be present in the gel at an amount sufficient to achieve a desired viscosity of the gel. In a particular instance, the amount of the rheological modifier can be between 20 wt. % to 30 wt. % of the total weight of the gel. The particulate sugar can be sucrose, maltose, lactose, etc., with sucrose being preferred. The surface treated silica can be silica silylate. The polyethylene glycol, in a preferred embodiment, can be PEG-8. The gel can include additional ingredients to add further moisturizing properties (e.g., jojoba esters), rheological properties (caprylyl glycol), and flavors (theobroma cacao (cocoa) shells, powders, etc.)) to the to the gel. In a particular embodiment, the gel has less than 5, 4, 3, 2, or 1 wt. % of vegetable oil, mineral, silicone oil, petrolatum, and petroleum oil. In one instance, the gel does not include any of vegetable oil, mineral, silicone oil, petrolatum, and petroleum oil. In still another instance, the gel can be as substantially depicted in Table 1 (with “substantially” meaning within 10% of the stated amounts of the ingredients identified in Table 1). Further, the dispersions of the present invention can be surfactant-free and still remain stable.

Also disclosed is a method of topically applying any one of the gels of the present invention to skin. The method can include topically applying the gel to skin (e.g., rubbing the gel on the skin with hands or an applicator (e.g., sponge or cloth). Due to the particulate sugar being suspended or dispersed throughout the gel matrix (the sugar is not solubilized in the gel matrix), the sugar can exfoliate or cleanses skin. The methods of the present invention therefore can include exfoliating or cleansing skin. Such methods can include first topically applying the gel to skin followed by rinsing the skin with water. Dirt, sebum, oil, dead skin cells, etc., along with the sugar scrub can be removed from the skin via rinsing with water. The gel can also be used to moisturize skin. The gel can be applied to dry skin, flaky skin, chapped skin, cracked skin, etc., to help moisturize such skin. The gel can be used on facial skin, and/or body skin (e.g., hands, arms, chest, abdomen, upper and lower back, legs, buttocks, feet, etc.).

In still another embodiment, there is disclosed a method of using any one of the aforementioned gels of the present invention to treat wounds (e.g., bed sores, diabetic ulcers, surgical incisions, skin burns, scratches, abrasions, etc.). The gels are particularly suited for the wound environment due to their sugar content, which can be used to treat wounds and speed up the wound healing process, and the fact that the gel does not require caustic materials to remain stable (e.g., surfactants and other cleansing agents). In this sense, an all-natural product can be used to treat wounds safely and effectively.

In yet another embodiment, there is a disclosed a method of treating or preventing a skin condition that can include topically applying any one of the gels disclosed throughout the specification to skin in need thereof. Non-limiting examples of such skin conditions include dry, cracked, or flaky skin (e.g., facial, scalp, hand, elbow, feet, heel, and other portions of skin that have a tendency to dry flake, or crack). Other conditions include fine lines or wrinkles, inflamed skin, erythemic skin, dead skin, sunburned skin, pruritus, spider veins, lentigo, age spots, senile purpura, keratosis, melasma, blotches, nodules, sun damaged skin, dermatitis (including, but not limited to seborrheic dermatitis, nummular dermatitis, contact dermatitis, atopic dermatitis, exfoliative dermatitis, perioral dermatitis, and stasis dermatitis), psoriasis, folliculitis, rosacea, acne, impetigo, erysipelas, erythrasma, eczema, and other inflammatory skin conditions. The skin can be facial skin or non-facial skin (e.g., arms, legs, hands, chest, back, feet, etc.). The method can further include identifying a person in need of skin treatment. The person can be a male or female. The age of the person can be at least 1, 2, 3, 4, 5, 6, 7, 8, 9, 10, 15, 20, 25, 30, 35, 40, 45, 50, 55, 60, 65, 70, 75, 80, 85, 90, 95, or more years old, or any range derivable therein. The method can also include topically applying an amount effective to: increase the stratum corneum turnover rate of the skin; increase collagen synthesis in fibroblasts; increase cellular anti-oxidant defense mechanisms (e.g., exogenous additions of anti-oxidants can bolster, replenish, or prevent the loss of cellular antioxidants such as catalase and glutathione in skin cells (e.g., keratinocytes, melanocytes, langerhans cells, etc.) which will reduce or prevent oxidative damage to the skin, cellular, proteins, and lipids); inhibit melanin production in melanocytes; reduce or prevent oxidative damage to skin (including reducing the amount lipid peroxides and/or protein oxidation in the skin).

Kits that include the gels of the present invention are also contemplated. In certain embodiments, the gel is included in a container. The container can be a bottle, dispenser, or package. The container can dispense a pre-determined amount of the dispersion. In certain aspects, the gel is dispensed in a spray, dollop, or liquid. The container can include indicia on its surface. The indicia can be a word, an abbreviation, a picture, or a symbol.

In the context of the present invention twenty-three (23) embodiments are disclosed. In one embodiment, a gel is disclosed. The gel can include a gel matrix that includes polyethylene glycol and surface treated silica, wherein the gel matrix is formed with the polyethylene glycol and surface treated silica; and particulate sugar dispersed throughout the gel matrix, wherein the gel has less than 5 wt. % oil. Embodiment 2 is the gel of embodiment 1 that can include 15 wt. % to 40 wt. % of polyethylene glycol and 1 wt. % to 5 wt. % of surface treated silica. Embodiment 3 is the gel of embodiment 2, that can include 30 wt. % to 70 wt. % of particulate sugar. Embodiment 4 is the gel of embodiment 3 that can include 20 wt. % to 30 wt. % of polyethylene glycol, 1 wt. % to 3 wt. % of surface treated silica, and 35 wt. % to 50 wt. % of particulate sugar. Embodiment 5 is the gel of embodiment 4 that can include a sugar alcohol. Embodiment 6 is the gel of embodiment 5, wherein the sugar alcohol is glycerol. Embodiment 7 is the gel of any one of embodiments 5 to 6 that can include 20 to 30 wt. % of the sugar alcohol. Embodiment 8 is the gel of any one of embodiments 1 to 7, wherein the particulate sugar is sucrose. Embodiment 9 is the gel of any one of embodiments 1 to 8, wherein the surface treated silica is silica silylate. Embodiment 10 is the gel of any one of embodiments 1 to 9, wherein the polyethylene glycol is PEG-8. Embodiment 11 is the gel of any one of embodiments 1 to 11 that can include jojoba esters, caprylyl glycol, and a flavoring agent. Embodiment 12 is the gel of embodiment 11, wherein the flavoring agent is theobroma cacao (cocoa) shell or powder or a combination thereof. Embodiment 13 is the gel of any one of embodiments 1 to 12, wherein the gel has less than 5 wt. % of vegetable oil, mineral, silicone oil, and petrolatum oil. Embodiment 14 is the gel of embodiment 13, wherein the gel does not include vegetable oil, mineral, silicone oil, and petrolatum oil. Embodiment 15 is the gel of embodiment 1, as substantially depicted in Table 1. Embodiment 16 is the gel of any one of embodiments 1 to 15, wherein the gel includes less than 5 wt. % of water.

Embodiment 17 is a method of topically applying any one of the gels of embodiments 1 to 16 to skin. The method can include topically applying the gel to skin. Embodiment 18 is the method of embodiment 17, wherein the gel exfoliates the skin. Embodiment 19 is the method of embodiment 18 that can include rinsing the gel from the skin with water, wherein dead skin cells are removed from the skin. Embodiment 20 is the method of any one of embodiments 17 to 19, wherein the gel moisturizes the skin. Embodiment 21 is the method of any one of embodiments 17 to 20, wherein the skin is dried, flaky, or cracked skin. Embodiment 22 is the method of embodiment 17, wherein the gel cleanses the skin. Embodiment 23 is the method of embodiment 22 that can include rinsing the gel from the skin with water, wherein dirt or sebum is removed from the skin.

It is contemplated that any embodiment discussed in this specification can be implemented with respect to any method or dispersion of the invention, and vice versa. Furthermore, dispersions of the invention can be used to achieve methods of the invention.

In one embodiment, the gels of the current invention are pharmaceutically elegant. “Pharmaceutically elegant” describes a dispersion that has particular tactile properties which feel pleasant on the skin (e.g., gels are not too watery or greasy and are not tacky or sticky, etc.). Pharmaceutically elegant can also relate to the creaminess or lubricity properties of the dispersion or to the moisture retaining properties of the gels.

“Keratinous tissue” includes keratin-containing layers disposed as the outermost protective covering of mammals and includes, but is not limited to, skin, hair and nails.

“Topical application” means to apply or spread a composition onto the surface of keratinous tissue. “Topical skin composition” and “dispersion” include compositions suitable for topical application on keratinous tissue. Such compositions are typically dermatologically-acceptable in that they do not have undue toxicity, incompatibility, instability, allergic response, and the like, when applied to skin. Dispersions of the present invention can have a selected viscosity to avoid significant dripping or pooling after application to skin.

“Exfoliating” means to remove dead or excess skin layers or cells from the surface of the skin.

“Surfactant-Free” means that the dispersion is free of surfactants. Surfactants include ingredients that have the ability to lower the surface tension of water or to reduce the interfacial tension between two immiscible substances. They are frequently classified as amphoteric, anionic, cationic, or nonionic surfactants.

“Oil” or “petroleum oil” as used herein refers to distillates of petroleum, silicone-based oils, or derivatives thereof. Non-limiting examples of oil or petroleum oil are distillates of petroleum, mineral oil, paraffinic oil, naphthenic oil, aromatic hydrocarbons (distinct form essential oils), petroleum wax and the like.

“Natural oil” as used herein refers to oils derived a plant (for example, from fruits, trees, bushes, nuts, vegetables, etc.).

“Physiologically acceptable agent” refers to an agent that is compatible with keratinous substances, such as the skin, nails, mucous membranes and keratinous fibers (for example, hair or eyelashes).

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The term “substantially” and its variations are defined as being largely but not necessarily wholly what is specified as understood by one of ordinary skill in the art, and in one non-limiting embodiment substantially refers to ranges within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the word “a” or “an” when used in conjunction with the term “comprising” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The gels of the present invention can “comprise,” “consist essentially of,” or “consist of” particular ingredients, components, compositions, etc. disclosed throughout the specification. In one aspect, gels consisting essentially of the claimed ingredients excludes ingredients that would materially affect the stability of the gels (e.g., cause the sugar phase to coalesce or cake during storage or use).

Other objects, features and advantages of the present invention will become apparent from the following detailed description, and examples. It should be understood, however, that the detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### DETAILED DESCRIPTION OF THE INVENTION

In today's image conscious society, people are continually looking for a product that can improve the visual appearance of their skin. For instance, symptoms associated with dry skin (e.g., flaky skin, dried or rough tactile quality, cracked skin, dehydrated skin, itchy skin, or red or erythemic skin) is associated with unattractive skin. Similarly, un-cleansed skin can be unsightly and can also lead to skin infections, lesions, pimples, acne, etc. Also, older aged, weathered, or damaged skin can be aesthetically unpleasing, and can be rejuvenated through exfoliation of the skin.

The inventor has discovered a stable gel-based sugar scrub that includes a gel matrix and particulate sugar suspended or dispersed throughout the gel matrix. The stability is achieved by creating a gel matrix from polyethylene glycol molecules and surface-treated silica. Without wishing to be bound by theory, it is believed that the surface treated silica particles interact with the polyethylene glycol molecules to produce a gel matrix that has the ability to keep high amounts of sugar particles suspended in the gel matrix. One benefit of such stability is that the end user of the gel of the present invention does not have to mix or shake the product before each use. This results in a consistent product being dispensed from the container, whereas compositions that have active ingredients that settle or coalesce can often times be widely inconsistent with each use (e.g., the dispensed product may have more or less of the active ingredient each time it is dispensed from the container). Further, the particulate sugar does not coalesce or cake in the bottle or when being applied to an end-user's skin.

These and other non-limiting aspects of the present invention are described in further detail below.

### A. Surface Treated Silica

The surface-treated silica is believed to act like a structuring agent when used in the gel-matrix. It is believed to provide rheological characteristics to the composition to contribute to the composition's stability and structure by promoting formation of a gel type structure when used in combination with the polyethylene glycol molecules. Silica is hydrophobic in nature and is treated with one or more organosilanes to produce the surface treated silica. Non-limiting examples, of organosilianes and organosiloxanes include organopolysiloxanes, hexamethyldisilazane, cyclotetramethyltetravinylsiloxane, and octylsilane. The surface treated silica can be in the form of an aerogel. In some aspects, the surface treated silica can be hydrophilic fumed silica that is further treated with an organosilane to produce a hydrophobic surface treated silica. In a preferred aspect, silica silylate is used. Surface treated silica are commercially available from Evonik Industries AG (Germany) as AEROSIL® R 805, AEROSIL® R 812, AEROSIL® R 812S, AEROSIL® R 816, AEROSIL® R 202, AEROSIL® R 972, AEROSIL® R 974. The surface treated silica can have a BET surface area from 100 to 180 m²/g, 120 to 170 m²/g, 130 to 160 m²/g, 140 to 150 m²/g.

### B. Polyethylene Glycols

Polyethylene glycol (PEG) molecules are polyether compounds having the general structure:

H—(O—CH₂—CH₂)ₙOH,

where n is such that the PEG molecule has a molecular mass below 10,000,000 g/mol, and preferably less than 20,000 g/mol. In addition to the above formula, PEGs can be branched or derivatized. Non-limiting examples of PEGs that can be used in the context of the present invention are disclosed in the International Cosmetic Ingredient Dictionary and Handbook, 12^{th} Edition, Volume 2, pages 1788-1995 (2008), which is incorporated herein by reference. In a preferred embodiment, PEG-8 is used, wherein n in the above formula is 8. However, other PEGs can be used (e.g., n is 4 to 800, such as PEG-4, PEG-6, PEG-7, PEG-9, PEG-10, PEG-100, . . . PEG-800, etc.). Still further, derivatives of PEGs can be used (e.g., PEG-8 avocadoate, PEG-8 amodimethicone, PEG-9 avocadoate, etc.).

### C. Particulate Sugar

The particulate sugar can be a mono or, more preferably, disaccharide sugar, most preferably sucrose, but could for example be fructose, maltose, glucose, invert sugar or a sugar alcohol. Other sugars, which can be used, include, for example, mannose, ribose, galactose, lactose, allose, altrose, talose, gulose, idose, arabinose, xylose, lyxose, erythrose, threose, acrose, rhamnose, fucose, glyceraldehyde, stachyose, agavose and cellobiose or a tri- or tetra-saccharide. The sugar agent can include granulated sucrose. Descriptions of sugar compounds can be found in Beet-Sugar Handbook (2007), by Mosen Asadi, PhD., and Sugar, A User's Guide to Sucrose (1990), by Neil L. Pennington and Charles W. Baker, both of which are incorporated into this specification by reference. Non-limiting examples of sugar alcohols include glycerol, erythritol, threitol, arabito, xylitol, ribitol, mannitol, sorbitol, galactitiol, fucitiol, iditol. In a preferred aspect, the sugar alcohol is glycerol.

### D. Petroleum Oils and Silicone Oils

Non-limiting examples of petroleum based oils or oils not derived from plants that are excluded from the composition include mineral oil, paraffinic oil, naphthenic oil, aromatic hydrocarbons, petroleum wax and/or silicone-based oils or silicone containing compounds. In non-limiting aspects, silicone containing compounds include any member of a family of polymeric products whose molecular backbone is made up of alternating silicon and oxygen atoms with side groups attached to the silicon atoms. By varying the —Si—O— chain lengths, side groups, and crosslinking, silicones can be synthesized into a wide variety of materials. They can vary in consistency from liquid to gel to solids. Non-limiting examples include silicone oils (e.g., volatile and non-volatile oils), gels, and solids. In certain aspects, the silicon containing compounds includes a silicone oils such as a polyorganosiloxane. Non-limiting examples of polyorganosiloxanes include dimethicone, cyclomethicone, polysilicone-11, phenyl trimethicone, trimethylsilylamodimethicone, stearoxytrimethylsilane, or mixtures of these and other organosiloxane materials in any given ratio in order to achieve the desired consistency and application characteristics depending upon the intended application (e.g., to a particular area such as the skin, hair, or eyes). A “volatile silicone oil” includes a silicone oil have a low heat of vaporization, i.e. normally less than about 50 cal per gram of silicone oil. Non-limiting examples of volatile silicone oils include: cyclomethicones such as Dow Corning 344 Fluid, Dow Corning 345 Fluid, Dow Corning 244 Fluid, and Dow Corning 245 Fluid, Volatile Silicon 7207 (Union Carbide Corp., Danbury, Conn.); low viscosity dimethicones, i.e. dimethicones having a viscosity of about 50 cst or less (e.g., dimethicones such as Dow Corning 200-0.5 cst Fluid). The Dow Corning Fluids are available from Dow Corning Corporation, Midland, Mich. Cyclomethicone and dimethicone are described in the Third Edition of the CTFA Cosmetic Ingredient Dictionary (incorporated by reference) as cyclic dimethyl polysiloxane compounds and a mixture of fully methylated linear siloxane polymers end-blocked with trimethylsiloxy units, respectively. Other non-limiting volatile silicone oils that can be used in the context of the present invention include those available from General Electric Co., Silicone Products Div., Waterford, N.Y. and SWS Silicones Div. of Stauffer Chemical Co., Adrian, Mich.

In addition to the combination of ingredients disclosed by the inventors, the compositions can also include additional ingredients such as cosmetic ingredients and pharmaceutical active ingredients. Non-limiting examples of these additional ingredients are described in the following subsections.

### E Cosmetic Ingredients

The CTFA International Cosmetic Ingredient Dictionary and Handbook (2004 and 2008) describes a wide variety of non-limiting cosmetic ingredients that can be incorporated into the gels of the present invention. Examples of these ingredient classes include: fragrances (artificial and natural), dyes and color ingredients (e.g., Blue 1, Blue 1 Lake, Red 40, titanium dioxide, D&C blue no. 4, D&C green no. 5, D&C orange no. 4, D&C red no. 17, D&C red no. 33, D&C violet no. 2, D&C yellow no. 10, and D&C yellow no. 11), adsorbents, lubricants, solvents, moisturizers (including, e.g., emollients, humectants, film formers, occlusive agents, and agents that affect the natural moisturization mechanisms of the skin), water-repellants, UV absorbers (physical and chemical absorbers such as para-aminobenzoic acid (“PABA”) and corresponding PABA derivatives, titanium dioxide, zinc oxide, etc.), essential oils, vitamins (e.g. A, B, C, D, E, and K), trace metals (e.g. zinc, calcium and selenium), anti-irritants (e.g. steroids and non-steroidal anti-inflammatories), botanical extracts (e.g. aloe vera, chamomile, cucumber extract, ginkgo biloba, ginseng, and rosemary), anti-microbial agents, antioxidants (e.g., BHT and tocopherol, cocoa powder), chelating agents (e.g., disodium EDTA and tetrasodium EDTA), preservatives (e.g., methylparaben and propylparaben), pH adjusters (e.g., sodium hydroxide and citric acid), absorbents (e.g., aluminum starch octenylsuccinate, kaolin, corn starch, oat starch, cyclodextrin, talc, and zeolite), skin bleaching and lightening agents (e.g., hydroquinone and niacinamide lactate), humectants (e.g., sorbitol, urea, methyl gluceth-20, and mannitol), exfoliants, waterproofing agents (e.g., magnesium/aluminum hydroxide stearate), skin conditioning agents (e.g., aloe extracts, allantoin, bisabolol, ceramides, dimethicone, hyaluronic acid, biosaccharide gum-1, ethylhexylglycerin, pentylene glycol, hydrogenated polydecene, octyldodecyl oleate, and dipotassium glycyrrhizate). Non-limiting examples of some of these ingredients are provided in the following subsections.

1. Moisturizing Agents

Non-limiting examples of other moisturizing agents that can be used with the compositions of the present invention include amino acids, chondroitin sulfate, diglycerin, glucose, glycerol polymers, glycols, 1,2,6-hexanetriol, honey, hyaluronic acid, hydrogenated honey, hydrogenated starch hydrolysate, inositol, lactitol, maltitol, maltose, mannitol, natural moisturizing factor, PEG-15 butanediol, polyglyceryl sorbitol, salts of pyrrolidone carboxylic acid, potassium PCA, caprylyl glycol, propylene glycol, sodium glucuronate, sodium PCA, sorbitol, sucrose, trehalose, urea, and xylitol.

Other examples include acetylated lanolin, acetylated lanolin alcohol, alanine, algae extract, aloe barbadensis, aloe-barbadensis extract, aloe barbadensis gel, althea officinalis extract, apricot (prunus armeniaca) kernel oil, arginine, arginine aspartate, arnica montana extract, aspartic acid, avocado (persea gratissima) oil, barrier sphingolipids, butyl alcohol, beeswax, behenyl alcohol, beta-sitosterol, birch (betula alba) bark extract, borage (borago officinalis) extract, butcherbroom (ruscus aculeatus) extract, butylene glycol, calendula officinalis extract, calendula officinalis oil, candelilla (euphorbia cerifera) wax, canola oil, caprylic/capric triglyceride, cardamom (elettaria cardamomum) oil, carnauba (copernicia cerifera) wax, carrot (daucus carota sativa) oil, castor (ricinus communis) oil, ceramides, ceresin, ceteareth-5, ceteareth-12, ceteareth-20, cetearyl octanoate, ceteth-20, ceteth-24, cetyl acetate, cetyl octanoate, cetyl palmitate, chamomile (anthemis nobilis) oil, cholesterol, cholesterol esters, cholesteryl hydroxystearate, citric acid, clary (salvia sclarea) oil, cocoa (theobroma cacao) butter, coco-caprylate/caprate, coconut (cocos nucifera) oil, collagen, collagen amino acids, corn (zea mays) oil, fatty acids, decyl oleate, dimethicone copolyol, dimethiconol, dioctyl adipate, dioctyl succinate, dipentaerythrityl hexacaprylate/hexacaprate, DNA, erythritol, ethoxydiglycol, ethyl linoleate, eucalyptus globulus oil, evening primrose (oenothera biennis) oil, fatty acids, geranium maculatum oil, glucosamine, glucose glutamate, glutamic acid, glycereth-26, glyceryl distearate, glyceryl hydroxystearate, glyceryl laurate, glyceryl linoleate, glyceryl myristate, glyceryl oleate, glyceryl stearate, glyceryl stearate SE, glycine, glycol stearate, glycol stearate SE, glycosaminoglycans, grape (vitis vinifera) seed oil, hazel (corylus americana) nut oil, hazel (corylus avellana) nut oil, hexylene glycol, hyaluronic acid, hybrid safflower (carthamus tinctorius) oil, hydrogenated castor oil, hydrogenated coco-glycerides, hydrogenated coconut oil, hydrogenated lanolin, hydrogenated lecithin, hydrogenated palm glyceride, hydrogenated palm kernel oil, hydrogenated soybean oil, hydrogenated tallow glyceride, hydrogenated vegetable oil, hydrolyzed collagen, hydrolyzed elastin, hydrolyzed glycosaminoglycans, hydrolyzed keratin, hydrolyzed soy protein, hydroxylated lanolin, hydroxyproline, hydrolyzed jojoba esters (simmondsia chinensis) isocetyl stearate, isocetyl stearoyl stearate, isodecyl oleate, isopropyl isostearate, isopropyl lanolate, isopropyl myristate, isopropyl palmitate, isopropyl stearate, isostearamide DEA, isostearic acid, isostearyl lactate, isostearyl neopentanoate, jasmine (jasminum officinale) oil, jojoba (buxus chinensis) oil, kelp, kukui (aleurites moluccana) nut oil, lactamide MEA, laneth-16, laneth-10 acetate, lanolin, lanolin acid, lanolin alcohol, lanolin oil, lanolin wax, lavender (lavandula angustifolia) oil, lecithin, lemon (citrus medica limonum) oil, linoleic acid, linolenic acid, macadamia ternifolia nut oil, maltitol, matricaria (chamomilla recutita) oil, methyl glucose sesquistearate, mink oil, mortierella oil, myristyl lactate, myristyl myristate, myristyl propionate, neopentyl glycol dicaprylate/dicaprate, octyldodecanol, octyldodecyl myristate, octyldodecyl stearoyl stearate, octyl hydroxystearate, octyl palmitate, octyl salicylate, octyl stearate, oleic acid, olive (olea europaea) oil, orange (citrus aurantium dulcis) oil, palm (elaeis guineensis) oil, palmitic acid, pantethine, panthenol, panthenyl ethyl ether, paraffin, PCA, peach (prunus persica) kernel oil, peanut (arachis hypogaea) oil, PEG-8 C12-18 ester, PEG-15 cocamine, PEG-150 distearate, PEG-60 glyceryl isostearate, PEG-5 glyceryl stearate, PEG-30 glyceryl stearate, PEG-7 hydrogenated castor oil, PEG-40 hydrogenated castor oil, PEG-60 hydrogenated castor oil, PEG-20 methyl glucose sesquistearate, PEG-40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (mentha piperita) oil, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, retinol, retinyl palmitate, rice (oryza sativa) bran oil, RNA, rosemary (rosmarinus officinalis) oil, rose oil, safflower (carthamus tinctorius) oil, sage (salvia officinalis) oil, sandalwood (santalum album) oil, serine, serum protein, sesame (sesamum indicum) oil, shea butter (butyrospermum parkii), silk powder, sodium chondroitin sulfate, sodium hyaluronate, sodium lactate, sodium palmitate, sodium PCA, sodium polyglutamate, soluble collagen, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, urea, vegetable oil, water, wheat (triticum vulgare) germ oil, and ylang ylang (cananga odorata) oil.

2. Antioxidants

Non-limiting examples of antioxidants that can be used with the compositions of the present invention include acetyl cysteine, ascorbic acid polypeptide, ascorbyl dipalmitate, ascorbyl methylsilanol pectinate, ascorbyl palmitate, ascorbyl stearate, BHA, BHT, t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, hydroquinone, isooctyl thioglycolate, kojic acid, magnesium ascorbate, magnesium ascorbyl phosphate, methylsilanol ascorbate, natural botanical anti-oxidants such as green tea or grape seed extracts, nordihydroguaiaretic acid, octyl gallate, phenylthioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and tris(nonylphenyl)phosphite.

3. Preservatives

Non-limiting examples of preservatives that can be used in the context of the present invention include quaternary ammonium preservatives such as polyquaternium-1 and benzalkonium halides (e.g., benzalkonium chloride (“BAC”) and benzalkonium bromide), phenoxyethanol, benzyl alcohol, caprylyl glycol, chlorobutanol, phenol, sorbic acid, thimerosal or combinations thereof.

4. Skin Lightening Agents

Non-limiting examples of skin lightening agents that can be used in the context of the present invention include dipotassium glycyrrhizate, ascorbyl glucoside, niacinamide, hydroquinone, or combination thereof.

5. UV Absorption or Sunscreen Agents

UV absorption or sunscreen agents that can be used in combination with the compositions of the present invention include chemical and physical sunblocks. Non-limiting examples of chemical sunblocks that can be used include para-aminobenzoic acid (PABA), PABA esters (glyceryl PABA, amyldimethyl PABA and octyldimethyl PABA), butyl PABA, ethyl PABA, ethyl dihydroxypropyl PABA, benzophenones (oxybenzone, sulisobenzone, benzophenone, and benzophenone-1 through 12), cinnamates (octyl methoxycinnamate, isoamyl p-methoxycinnamate, octylmethoxy cinnamate, cinoxate, diisopropyl methyl cinnamate, DEA-methoxycinnamate, ethyl diisopropylcinnamate, glyceryl octanoate dimethoxycinnamate and ethyl methoxycinnamate), cinnamate esters, salicylates (homomethyl salicylate, benzyl salicylate, glycol salicylate, isopropylbenzyl salicylate, etc.), anthranilates, ethyl urocanate, homosalate, octisalate, dibenzoylmethane derivatives, octyl triazone, digalloy trioleate, glyceryl aminobenzoate, lawsone with dihydroxyacetone, ethylhexyl triazone, dioctyl butamido triazone, benzylidene malonate polysiloxane, terephthalylidene dicamphor sulfonic acid, disodium phenyl dibenzimidazole tetrasulfonate, diethylamino hydroxybenzoyl hexyl benzoate, bis diethylamino hydroxybenzoyl benzoate, bis benzoxazoylphenyl ethylhexylimino triazine, drometrizole trisiloxane, methylene bis-benzotriazolyl tetramethylbutyiphenol, and bis-ethylhexyloxyphenol methoxyphenyltriazine, 4-methylbenzylidenecamphor, and isopentyl 4-methoxycinnamate. Non-limiting examples of physical sunblocks include, kaolin, talc, petrolatum and metal oxides (e.g., titanium dioxide and zinc oxide).Compositions of the present invention can have UVA and UVB absorption properties. The compositions can have an sun protection factor (SPF) of 2, 3, 4, 56, 7, 8, 9, 10, 11, 12, 13, 14, 15, 20, 25, 30, 35, 40, 45, 50, 55, 60, 70, 80, 90 or more, or any integer or derivative therein.

6. Thickening Agents

Thickening agents, including thickener or gelling agents, include substances which that can increase the viscosity of a composition. Thickeners includes those that can increase the viscosity of a composition without substantially modifying the efficacy of the active ingredient within the composition. Thickeners can also increase the stability of the compositions of the present invention. In certain aspects of the present invention, thickeners include hydrogenated polyisobutene, trihydroxystearin, ammonium acryloyldimethyltaurate/vp copolymer, or a mixture of them.

Non-limiting examples of additional thickening agents that can be used in the context of the present invention include carboxylic acid polymers, crosslinked polyacrylate polymers, polyacrylamide polymers, polysaccharides, and gums. Examples of carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol (see U.S. Pat. Nos. 5,087,445; 4,509,949; 2,798,053; CTFA International Cosmetic Ingredient Dictionary, Fourth edition, 1991, pp. 12 and 80). Examples of commercially available carboxylic acid polymers include carbomers, which are homopolymers of acrylic acid crosslinked with allyl ethers of sucrose or pentaerytritol (e.g., Carbopol™ 900 series from B. F. Goodrich).

Non-limiting examples of crosslinked polyacrylate polymers include cationic and nonionic polymers. Examples are described in U.S. Pat. Nos. 5,100,660; 4,849,484; 4,835,206; 4,628,078; 4,599,379).

Non-limiting examples of polyacrylamide polymers (including nonionic polyacrylamide polymers including substituted branched or unbranched polymers) include polyacrylamide, isoparaffin and laureth-7, multi-block copolymers of acrylamides and substituted acrylamides with acrylic acids and substituted acrylic acids.

Non-limiting examples of polysaccharides include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Another example is an alkyl substituted cellulose where the hydroxy groups of the cellulose polymer is hydroxyalkylated (preferably hydroxy ethylated or hydroxypropylated) to form a hydroxyalkylated cellulose which is then further modified with a C10 -C30 straight chain or branched chain alkyl group through an ether linkage. Typically these polymers are ethers of C10-C30 straight or branched chain alcohols with hydroxyalkylcelluloses. Other useful polysaccharides include scleroglucans that include a linear chain of (1-3) linked glucose units with a (1-6) linked glucose every three unit.

Non-limiting examples of gums that can be used with the present invention include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

F. Pharmaceutical Ingredients

Pharmaceutical ingredients are also contemplated as being useful with the emulsion compositions of the present invention. Non-limiting examples of pharmaceutical ingredients include anti-acne agents, agents used to treat rosacea, analgesics, anesthetics, anorectals, antihistamines, anti-inflammatory agents including non-steroidal anti-inflammatory drugs, antibiotics, antifungals, antivirals, antimicrobials, anti-cancer actives, scabicides, pediculicides, antineoplastics, antiperspirants, antipruritics, antipsoriatic agents, antiseborrheic agents, biologically active proteins and peptides, burn treatment agents, cauterizing agents, depigmenting agents, depilatories, diaper rash treatment agents, enzymes, hair growth stimulants, hair growth retardants including DFMO and its salts and analogs, hemostatics, kerotolytics, canker sore treatment agents, cold sore treatment agents, dental and periodontal treatment agents, photosensitizing actives, skin protectant/barrier agents, steroids including hormones and corticosteroids, sunburn treatment agents, sunscreens, transdermal actives, nasal actives, vaginal actives, wart treatment agents, wound treatment agents, wound healing agents, etc.

G. Method of Making the Gel

In addition to the methods disclosed in the Examples section of this specification, another non-limiting method for making a gel of the present invention includes: (1) obtain the surface treated silica and mix the surface treated silica with an emulsifying agent until a gel is formed. A portion of the sugar agent (for example, the sugar alcohol and a portion of the sugar) is added to the gel. The moisturizing and other ingredients can be obtained and mixed with the mixture of the gel and sugar agent. The remaining sugar agent (for example, sucrose) can be added to form the topical dispersion of the invention. The gel is ready to be used and can be used in any manner described throughout this specification.

H. Kits

Kits are also contemplated as being used in certain aspects of the present invention. For instance, compositions of the present invention can be included in a kit. A kit can include a container. Containers can include a bottle, a metal tube, a laminate tube, a plastic tube, a dispenser, a pressurized container, a barrier container, a package, a compartment, a lipstick container, a compact container, cosmetic pans that can hold cosmetic compositions, or other types of containers such as injection or blow-molded plastic containers into which the dispersions or compositions or desired bottles, dispensers, or packages are retained. The kit and/or container can include indicia on its surface. The indicia, for example, can be a word, a phrase, an abbreviation, a picture, or a symbol.

The containers can dispense a pre-determined amount of the composition. In other embodiments, the container can be squeezed (e.g., metal, laminate, or plastic tube) to dispense a desired amount of the composition. The composition can be dispensed as a spray, an aerosol, a liquid, a fluid, or a semi-solid. The containers can have spray, pump, or squeeze mechanisms. A kit can also include instructions for employing the kit components as well the use of any other compositions included in the container. Instructions can include an explanation of how to apply, use, and maintain the compositions.

### EXAMPLES

The following examples are included to demonstrate certain non-limiting aspects of the invention. It should be appreciated by those of skill in the art that the techniques disclosed in the examples which follow represent techniques discovered by the inventor to function well in the practice of the invention. However, those of skill in the art should, in light of the present disclosure, appreciate that many changes can be made in the specific embodiments which are disclosed and still obtain a like or similar result without departing from the spirit and scope of the invention.

### Example 1

### Gel-Based Sugar Scrub

Table 1 provides a non-limiting example of a gel-based sugar scrub of the present invention. It was discovered that the use of surface treated silica (silica silylate) with polyethylene glycol creates a gel matrix that allows for a high amount of sugar to be present in the matrix than would otherwise expected. Without wishing to be bound by theory, it is believed that the silica silylate helps keep the sugar suspended throughout the gel matrix. The dispersion remained stable (no coalescence or “caking” of sugar) under stability testing conditions. The remaining ingredients provided tactile properties and rheological properties to the gelled dispersion. Notably, the stable dispersion is substantially oil free (e.g., less than 5 wt. % oil, preferably 2 wt. % or less oil such as jojoba esters).

TABLE 1*

| | | Amount by |
|---|---|---|
| Phase | Ingredient | weight (grams) |
| A | PEG-8 | 26.25 |
| A | Silica silylate** | 1.8 |
| B | Glycerol (99.5%) | 26.25 |
| C | Jojoba esters*** | 2.0 |
| C | Sucrose | 2.0 |
| C | Theobroma Cacao (Cocoa) Shell | 1.0 |
| D | Caprylyl glycol | 0.5 |
| D | Cocoa Powder | 0.2 |
| E | Sucrose | 40.0 |
| | Total | 100 |

*Formulation was prepared as follows (1) weighed ingredients; (2) mixed the polyethylene glycol propylene glycol cocoate and silica silylate in a container and with a spatula until mixture was thoroughly blended (gel phase A); (3) To phase A, the physiological acceptable agent ingredients were added in the following order (1) add glycerol and heat. (2) added simmondsia chinensis and, sucrose and heat until simmondsia chinensis is dissolved (4) added theobroma cacao, caprylyl glycol and cocoa powder and cool to room temperature (5) added fragrance and sucrose and mixed with a spatula until the sucrose was dispersed throughout the dispersion.
**Obtained from Evonik Industries AG (Germany) under the trade name Aerosil ® R 805 (hydrophobic fumed silica).
***Obtained from FloraTech (Chandler, Arizona USA) under the trade name Floraesters ® K-100 Jojoba (mixture of hydrolyzed jojoba esters (75-85%), nonhydrolyzed jojoba esters (8-15%), and water (7 to 10%).

1. A gel comprising:
(a) a gel matrix comprising polyethylene glycol and surface treated silica, wherein the gel matrix is formed with the polyethylene glycol and surface treated silica; and
(b) particulate sugar dispersed throughout the gel matrix,
wherein the gel has less than 5 wt. % oil.

2. The gel of claim 1, comprising 15 wt. % to 40 wt. % of polyethylene glycol and 1 wt. % to 5 wt. % of surface treated silica.

3. The gel of claim 2, comprising 30 wt. % to 70 wt. % of particulate sugar.

4. The gel of claim 3, comprising 20 wt. % to 30 wt. % of polyethylene glycol, 1 wt. % to 3 wt. % of surface treated silica, and 35 wt. % to 50 wt. % of particulate sugar.

5. The gel of claim 4, further comprising a sugar alcohol.

6. The gel of claim 5, wherein the sugar alcohol is glycerol.

7. The gel of claim 5, comprising 20 to 30 wt. % of the sugar alcohol.

8. The gel of claim 1, wherein the particulate sugar is sucrose.

9. The gel of claim 1, wherein the surface treated silica is silica silylate.

10. The gel of claim 1, wherein the polyethylene glycol is PEG-8.

11. The gel of claim 1, further comprising jojoba esters, caprylyl glycol, and a flavoring agent.

12. The gel of claim 11, wherein the flavoring agent is theobroma cacao (cocoa) shell or powder or a combination thereof.

13. The gel of claim 1, wherein the gel has less than 5 wt. % of vegetable oil, mineral, silicone oil, and petrolatum oil.

14. The gel of claim 13, wherein the gel does not include vegetable oil, mineral, silicone oil, and petrolatum oil.

15. The gel of claim 1, as substantially depicted in Table 1.

16. The gel claim 1, wherein the gel includes less than 5 wt. % of water.

17. A method of topically applying the gel of claim 1 to skin, the method comprising topically applying the gel to skin.

18. The method of claim 17, wherein the gel exfoliates the skin.

19. The method of claim 18, further comprising rinsing the gel from the skin with water, wherein dead skin cells are removed from the skin.

20. The method of claim 17, wherein the gel moisturizes the skin.

21. The method of claim 17, wherein the skin is dried, flaky, or cracked skin.

22. The method of claim 17, wherein the gel cleanses the skin.

23. The method of claim 22, further comprising rinsing the gel from the skin with water, wherein dirt or sebum is removed from the skin.
